(19) United States
(12) Patent Application Publication
(10) Pub. No.: US 2014/0025050 A1
(43) Pub. Date: Jan. 23, 2014

(54) VACUUM SUCTION PRESSURE DEVICE EQUIPPED WITH HEAT ENABLED INSERT

(71) Applicant: Robert S. Anderson, Livermore, CA (US)

(72) Inventor: Robert S. Anderson, Livermore, CA (US)

(73) Assignee: Theradyme, Inc., Livermore, CA (US)

(21) Appl. No.: 13/551,689

(22) Filed: Jul. 18, 2012

(51) Int. Cl. A61B 18/18; A61B 18/20; A61B 18/04

(52) U.S. Cl. 606/9; 606/28; 606/33

(57) ABSTRACT

Methods and an apparatus for heating up a surface of a skin area that is to undergo a topical treatment are disclosed. A method placing a device enabled with vacuum suction pressure on a surface of a tissue area to be treated. Applying a vacuum suction pressure on the surface of the skin area to pull up the skin area, and an underlying tissue into an aperture opening of the device Simultaneously, while retaining vacuum suction, heating up a volume of tissue that is pulled up inside the aperture opening of the device such that the temperature of the tissue area rises to an elevated ambient temperature and performing a desired treatment, through an energy-generating module, on the tissue volume after the tissue area has been heated up.

### FIELD OF INVENTION

This disclosure relates generally to the field of medical devices more particularly to devices used to treat medical conditions through the skin.

### BACKGROUND

Millions of American teenagers and adults suffer from conditions that require treatments administered through the skin. For example, millions of Americans suffer from acne, excess hair, scars, blemishes, cellulite and other such conditions. The conditions requiring treatment may be administered through a medical device that may need to penetrate deep under the surface of the skin. The condition may require administration of heat in the form of light or other forms of energy to the target area to be treated. For example, in the case of laser hair removal, medical personnel may need to administer enough heat to the area to destroy the hair follicle without damaging the surrounding tissue. However, delivering heat to a localized part of the body may be tricky and dangerous, and may have the effect of permanently damaging the targeted tissue area or surrounding tissue areas if not administered correctly. Also, the skin may be very sensitive to excess heat and may burn, or suffer from hyperpigmentation or hypopigmentation without proper administration of the heat energy. As a consequence, medical personnel may not be able to increase the amount of heat energy delivered to the area that needs to be treated, in order to prevent such extreme risks to the surrounding tissue area. Therefore, the efficacy of the treatment may be compromised. Additionally, the cost of administering heat to the area of skin may be unreasonable high and inefficient and may unnecessarily drive up treatment costs. The lack of effective treatment options having immediate efficacy may be frustrating and stressful for patients who are already taxed with having a constantly recurring condition like the ones named above. Furthermore, patients may spend large amounts of money on ineffective treatments or even risk self-treatment by dangerous and ineffective means.

### SUMMARY

Disclosed are a method, an apparatus and/or a system of treating skin or underlying conditions in a safe and cost effective manner. In one aspect, the method includes placing a device enabled with vacuum suction pressure on a surface of a tissue area to be treated, applying a vacuum suction pressure on the surface of the skin area to pull up the skin area and an underlying tissue into an aperture opening of the device, simultaneously, while retaining vacuum suction, heating up a volume of tissue that is pulled up inside the aperture opening of the device such that the temperature of the tissue area rises to an elevated ambient temperature and performing a desired treatment, through an energy-generating module, on the tissue volume after the tissue area has been heated up.

In another aspect, an apparatus is disclosed that includes a device enabled with vacuum suction pressure to enclose a volume of tissue area that is to undergo a desired treatment, to pull in the volume of the tissue area into an aperture of the device when vacuum suction pressure is applied, to release an application of the vacuum suction pressure at a time that marks at least one of before a start of the desired treatment and a completion of the desired treatment. The apparatus may also include a set of heating elements to simultaneously heat up the volume of tissue that is pulled inside the aperture such that the temperature of the tissue area rises to an elevated ambient temperature. The apparatus may also include an energy generating module to deliver at least one of a light and a source of heat energy to the tissue area to after the volume of tissue after the temperature of the tissue has risen to the elevated ambient temperature from a normal body basal temperature.

In yet another aspect, a method is disclosed that includes applying a vacuum suction pressure on the surface of the skin area to pull up the skin area and an underlying tissue into an aperture opening of the device, simultaneously, while retaining vacuum suction, heating up a volume of tissue that is pulled up inside the aperture opening of the device such that the temperature of the tissue area rises to an elevated ambient temperature, performing a desired treatment, through an energy-generating module, on the tissue volume after the tissue area has been heated up, and releasing the application of the vacuum suction pressure on the surface of the tissue area at a time that marks at least one of before a start of the desired treatment and a completion of the desired treatment.

### BRIEF DESCRIPTION OF FIGURES

Example embodiments are illustrated by way of example and not limitation in the figures of the accompanying drawings, in which like references indicate similar elements and in which:

FIG. 1 is a drawing showing a medical device that may be used to treat a condition through the skin being used by a medical personnel on a patient.

FIG. 2 illustrates the medical device with the various modules in the device that may be used to treat a medical condition in the patient.

FIGS. 3A and 3B is a process flow diagram illustrating how the medical device fitted with a heat insert works to heat up the tissue to be treated.

FIG. 4 is a close-up view of the medical device when the skin is being pulled into an aperture of the medical device. FIG. 5 is a process flow diagram illustrating how the medical device fitted with a needle insert works to treat a condition in the patient.

FIG. 6 is a system architecture view, specifically detailing the vacuum suction module.

### DETAILED DESCRIPTION

Disclosed are a method, an apparatus and/or a system of treating skin or underlying condition in a safe and cost effective manner In the following description, for the purposes of explanation, numerous specific details are set forth in order to provide a thorough understanding of the various embodiments. It will be evident, however, to one skilled in the art that the various embodiments may be practiced without these specific details.

Detailed Background and Causes

Many medical treatments may need to be performed by supplying energy through the epidermis. Such treatments may pertain only to a localized part of the body whose effects are only applied to the localized part of the body and may not affect other parts of the body to which the treatment is applied. Many conditions are treated in this manner so as to only target the area of the body that may require treatment. Examples of conditions that are typically treated in this manner may include acne, pigmented lesion removal, vascular lesion removal, cellulite reduction treatments, tattoo removal treatments, hair removal treatments and others.

Treatments that supply energy through the epidermis may be desirable in many cases because the treatment may only affect the area to be treated without affecting the entire body. For these treatments to be effective, the treatment may need to be severely localized with high accuracy and efficacy. An ideal way to treat some medical conditions may be through a medical device that provides cost effective treatment through the skin without damaging the skin and the underlying tissue. Some examples of localized treatments delivered through the skin are detailed below.

Light Therapy

Many medical devices may employ the use of light therapy or phototherapy to treat various epidermal and dermal conditions and other underlying medical conditions. Light therapy or phototherapy may consist of exposure to broadband light or specific wavelengths of light. These may be administered through the use of lasers, light emitting diodes, fluorescent lamps, flash lamps, or very bright, full-spectrum light, usually controlled with various devices. In one or more embodiments, the use of light also has the effect of heating up the targeted area of tissue. The use of light to generate heat may be effective to treat various epidermal and dermal conditions, but may need to be regulated carefully to avoid burning or damaging the epidermis area and the underlying tissue. Also, the administration of light may be expensive, which in turn, may drive up costs of the treatment.

Needle Therapy

Many medical devices may employ the use of a single needle or an array of needles to treat various epidermal and dermal conditions and other underlying medical conditions. The treatment energy may be administered through the use of radio frequency usually controlled with various devices. In one or more embodiments, the use of radio frequency energy also has the effect of heating up the targeted area of tissue. The use of radio frequency to generate heat may be effective to treat various epidermal and dermal conditions, but may need to be regulated carefully to avoid burning or damaging the epidermis and dermis of the underlying tissue. Also, the administration of radio frequency may be expensive, which in turn, may drive up costs of the treatment.

Ultrasound Therapy

Many medical devices may employ the use of an ultrasound to treat various epidermal and dermal conditions and other underlying medical conditions. The treatment energy may be administered through the use of ultrasound energy usually controlled with various devices. In one or more embodiments, the use of ultrasound energy also has the effect of heating up the targeted area of tissue. The use of ultrasound to generate heat may be effective to treat various epidermal and dermal conditions, but may need to be regulated carefully to avoid burning or damaging the epidermis and dermis of the underlying tissue. Also, the administration of ultrasound may be expensive, which in turn, may drive up costs of the treatment.

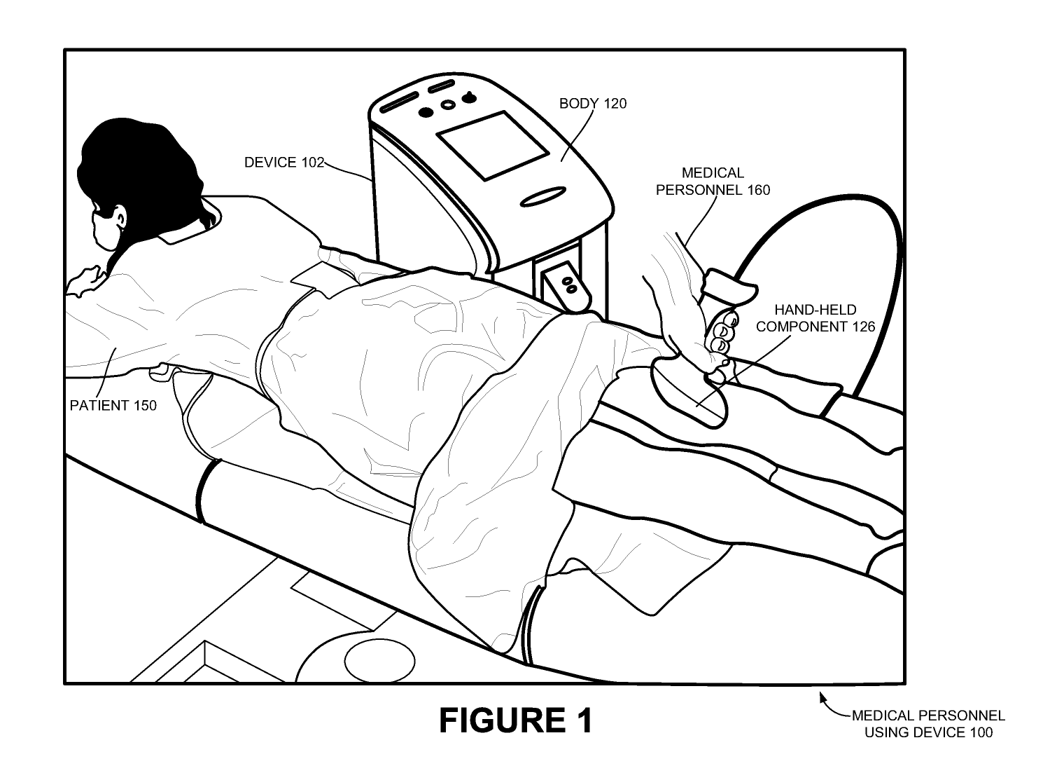

FIG. 1 is a drawing showing a medical device that may be used to treat a condition through the skin. FIG. 1 shows the body 120 of the device 102 and the hand-held component 126 of the device, the medical personnel 160 and the patient 150.

In one or more embodiments, a single medical device may be able to administer multiple types of treatments through the skin. In one or more embodiments, the medical device may have multiple interchangeable inserts and modules to perform a wide variety of treatments. Treatments may be officially performed by a medical personnel or a doctor on a desired treatment area for a patient coming in for a certain type of treatment. For example, as shown in FIG. 1, the patient 150 may be being treated by the medical personnel 160 for a hair removal procedure on her leg.

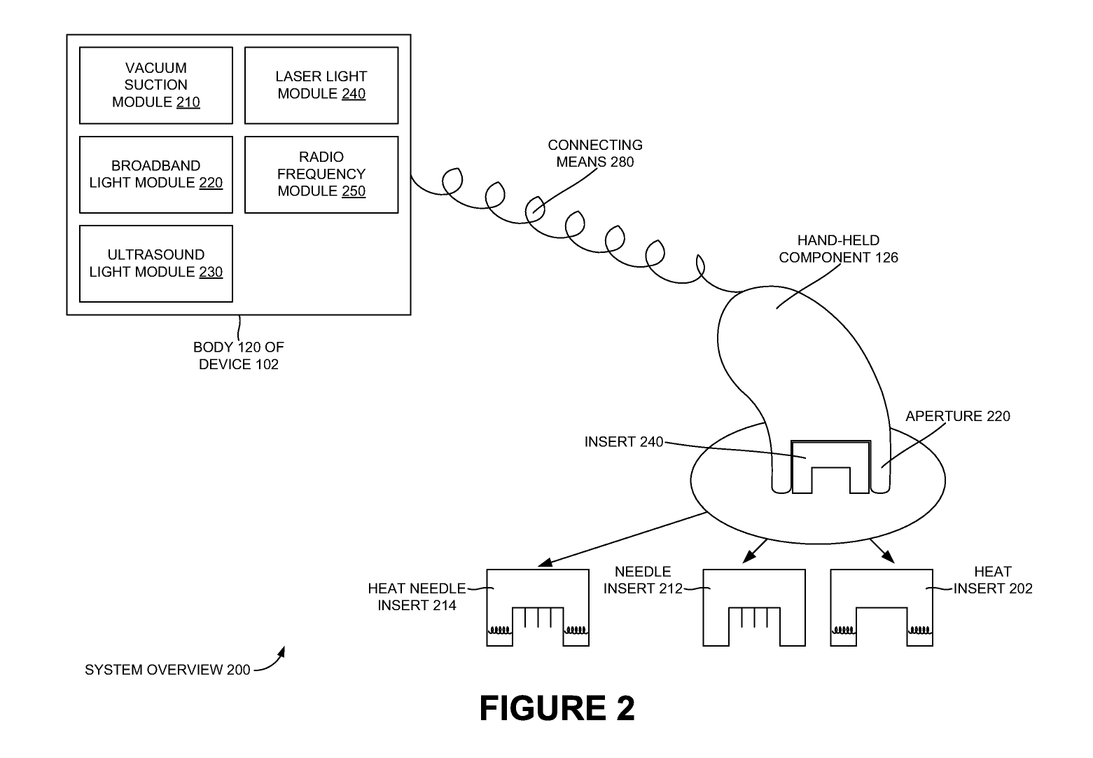

In one or more embodiments, the medical device may have a probe or hand-held treatment component and a body. The body 120 may contain the electronics and electrical components of the medical device as will be described below. The hand-held treatment component 126 may be the part of the medical device that may be used by the medical personnel to administer the treatment to the desired treatment area. In one or more embodiments, the body may be connected to the hand-held treatment component 126 through an electrical wire or other connecting means. In one or more embodiments, the hand-held component 126 may contain an aperture as shown in FIG. 2. The aperture may be a recessed section of the hand-held component through which the treatment (vacuum, light or heat energy) may be administered to the desired treatment area, in one or more embodiments.

FIG. 2 illustrates a range of functions of the medical device and includes a vacuum suction module 210, a laser light module 240, a broadband light module 220, a radio frequency module 250, and an ultrasound light module 230 which may all reside in the body 120 of the device 102 or in the hand held component 126. In one or more embodiments, all the above mentioned modules may be considered energy generating modules. In one or more embodiments, the vacuum suction pressure device 102 may contain one or more heating elements and a source of light energy. This light energy may be filtered using an optical element. In other words, any of these above mentioned energy generating modules may be used to perform a desired treatment on a skin area of the patient 150. The hand held component 126 of the device 102 may have an aperture 220 as shown in FIG. 2. The aperture 220 may be fitted in with an insert. An insert may snugly fit into the aperture 220 of the device 102 and may be used to complement the treatment as will be described in the application later. Two examples of possible inserts may be the needle insert 212 and the heat insert 202. The heat insert 202 may contain a pair of electrodes on the sides of the heat insert as shown in the Figure. The needle insert 212 may contain at least one needle or micro-needle recessed into the insert as shown in the Figure. The insert may also be a heated needle insert, or a heat needle insert as will be described below as well.

In one or more embodiments, the medical device may have at least one of a vacuum suction module, a broadband light module, an ultrasound light module, a heat generating module, a laser module. In one or more embodiments, the medical device may be equipped with all the above mentioned modules. In one or more embodiments, the medical device may only contain one or two modules. For example, the medical device may be equipped with only the vacuum suction module and the laser module if the medical device is solely to be used for laser hair removal, for instance.

The vacuum suction module 210 may generate a vacuum of less than 7 psi such that when the aperture of the medical device is placed on the surface of the skin area to be treated, the light suction generated by the vacuum suction module pulls up the tissue area into the aperture of the medical device. The magnitude of vacuum suction generated by the vacuum suction module of the medical device may be modified by the medical personnel as necessary. When the vacuum suction is turned on, the skin and the underlying tissue may be pulled up into the aperture of the device. When the vacuum suction is turned off, the skin is released from the aperture of the medical device.

The broad band light module 220 may generate broad band light through the aperture of the medical device, in one or more embodiments. The magnitude of the broad band light may be altered or modified by the medical personnel as necessary. In one or more embodiments, the broad band light may have the effect of generating heat to the targeted area, and may heat up the targeted area to a desired temperature. In one or more embodiments, the broad band light and vacuum suction may work in concert such that the tissue is first pulled into the aperture first by the vacuum suction module, and then the broad band light is generated through the aperture as well. After the broad band light has been administered to the targeted area for the desired treatment time, the broad band light may be turned off and the vacuum suction may be turned off such that the skin is restored to its previous condition.

The laser module 240 may generate laser light through the aperture of the medical device, in one or more embodiments. Laser light of a specified wavelength, as needed for the medical procedure may be generated through the aperture of the device to the targeted tissue area, in one or more embodiments. The laser light may generate a specific amount of heat energy based on the wavelength of the laser light. This amount of heat energy may be used for the desired treatment, in one or more embodiments. Since laser light is specific to a particular wavelength medical personnel can deliver a precise amount of energy to the targeted tissue area. The medical personnel may be able to modify the wavelength of the laser light in one or more embodiments. The range of wavelengths of the laser module may be from 400 nm to 10.6 microns. In one or more embodiments, the laser module and the vacuum suction module may work in concert such that the tissue is first pulled into the aperture first by the vacuum suction module, and then the laser light of the desired wavelength is generated through the aperture as well. After the laser has been administered to the targeted area for the desired treatment time, the laser may be turned off and the vacuum suction may be turned off such that the skin is restored to its previous condition.

The ultrasound module 230 may generate ultrasound energy through the aperture of the medical device, in one or more embodiments. Ultrasound energy of a desired magnitude as needed for the medical procedure may be generated through the aperture of the device to the targeted tissue area, in one or more embodiments. The medical personnel may be able to modify the magnitude of energy in one or more embodiments. In one or more embodiments, the ultrasound module and the vacuum suction module may work in concert such that the tissue is first pulled into the aperture first by the vacuum suction module, and then the ultrasound energy of the desired magnitude is generated through the aperture as well. After the energy has been administered to the targeted area for the desired treatment time, the ultrasound may be turned off and the vacuum suction may be turned off such that the skin is restored to its previous condition.

The radio frequency module 250 may generate radio frequency energy through the aperture of the medical device, in one or more embodiments. Radio frequency energy of a desired magnitude as needed for the medical procedure may be generated through the aperture of the device to the targeted tissue area, in one or more embodiments. The medical personnel may be able to modify the magnitude of energy in one or more embodiments. The range of delivered energy of the radio frequency module may be 0.5 Joules to 50 Joules. In one or more embodiments, the radio frequency module and the vacuum suction module may work in concert such that the tissue is first pulled into the aperture first by the vacuum suction module, and then the radio frequency energy of the desired magnitude is generated through the aperture as well. After the energy has been administered to the targeted area for the desired treatment time, the radio frequency module may be turned off and the vacuum suction may be turned off such that the skin is restored to its previous condition.

In one or more embodiments, the aperture 220 of the medical device 102 may be fitted with an insert. The insert may be a removable component that may fit exactly into the aperture of the medical device. The type of insert affixed to the aperture of the medical device may depend on the type of treatment desired. The specifications of the insert may be exactly the size of the aperture of the medical device such that the insert fits snugly into the recessed portion of the aperture. The insert may have at least one hole on the top portion of the insert that is immediately touching the top part of the aperture such that the various modules described above may work perfectly even with the insert recessed into the aperture. In one or more embodiments, the insert may be designed with an area such that the insert fits in exactly into the aperture 220 of the vacuum suction pressure device 102. In one or more embodiments, the insert may snap into the aperture 220 of the vacuum suction device 102. In one or more embodiments, part of the insert (the part of the insert that snaps into the aperture 220 of the vacuum suction device 102) may have holes such that the vacuum generated by the vacuum suction device 102 is still able to function through the insert.

In one or more embodiments, a heat insert may be fitted into the aperture of the device. The heat insert as will be described in detail below may have a heating section around the periphery of the inside section of the insert, in one or more embodiments. In one or more embodiments, a set of heating elements may be located on a periphery of the inside section of the insert. The inside section of the insert may be the part of the insert that comes into direct contact with the tissue. In one or more embodiments, the heating elements may be recessed into the aperture such that the heating elements do not touch the surface of the skin area unless the volume of tissue has been pushed into the aperture. In one or more embodiments, the heating elements may be electrodes capable of conducting electricity through the volume of tissue located between the two electrodes. In one or more embodiments, the heat insert may be used with the vacuum suction module such that when the skin is pulled up into the aperture of the device, a set of electrodes in the heating section heat up the volume of tissue pulled into the aperture. In one or more embodiments, the heat insert may also be used in concert with any other module as well, such that the heat insert heats up the volume of tissue pulled in into the aperture of the device and any energy generated by the other modules described above may administer energy to the targeted tissue area as well. This dual heating force(with the heat insert) may have the effect of preheating the targeted tissue area, such that the amount of energy needed to elevate temperature of the underlying tissue may be less than what would be required without the heat insert. For example, in the case of laser hair removal procedure, the tissue area may need to be heated up to 65 degree C. to successfully destroy the hair follicle. Without the heat insert, the laser module of 810 nm wavelength may need to deliver an energy density of 30 J/cm2 However, with the heat insert, the tissue area may already be heated up to the elevated ambient temperature of 45 degree C., such that the laser module of 810 nm wavelength may only need to deliver an energy density of 15 J/cm2. This reduced dosage of energy may be gentler on the skin and may reduce the risks of damaging the surrounding tissue area. Also, generating laser light may be very expensive. Therefore, reducing the amount of laser light required for the treatment may drive down treatment costs for such procedures. The heat insert and its practical usage will be described in further detail below.

In one or more embodiments, a needle insert may be fitted into the aperture as well. The needle insert may contain at least one needle recessed into the insert such that when the surface of the skin is pulled into the aperture of the device, the needles puncture the tissue to create a lesion on the surface of the skin area. The needle insert may be used in concert with the vacuum suction pressure device to pull up the tissue area into the aperture and create lesions on the surface of the skin. In one or more embodiments, the needle insert may be removed, and then the aperture of the medical device may then be used on the newly punctured skin to administer light or heat to the targeted tissue area.

In one or more embodiments the needles may be recessed such that the length of the needles is smaller than the length of a depth of the needle insert. In other words, the needles would only be able to touch a surface that is pulled into the depth of the needle insert. In one or more embodiments, the needle insert 212 may be designed with an area and/or length/width such that the needle insert 212 fits exactly into an aperture 220 of the vacuum suction pressure device 102. In one or more embodiments, the needle insert 103 snaps into the aperture 220 of the vacuum suction pressure device 102. In one or more embodiments, the top portion of the needle insert 212 may have holes such that the vacuum generated by the vacuum suction pressure device 102 is still able to function through the needles. In other words, the vacuum pressure generated by the device 102 is able to function and pull even with the presence of the needle insert 212.

In one or more embodiments, a heated needle insert may be fitted into the aperture. The needles of the needle insert may first be heated to a desired temperature and then the vacuum suction might be applied such that when the tissue area is pulled up into the aperture of the device, the needles create a lesion into the skin area and also have the effect of heating up the volume of the tissue area with the heated needles as well. In one or more embodiments, after the tissue is heated and the lesions are created, the device, through one of the other modules may then deliver heat energy to the newly punctured skin. As mentioned above, the heat may then be able to penetrate deeper into the tissue area, and may also require a smaller dosage of energy applied to the skin.

In one or more embodiments, a heat-needle insert may be fitted into the aperture of the medical device. In one or more embodiments, the heat needle insert may have at least one needle recessed into the aperture and may also have a heating section around the periphery of the inside section of the insert. When the skin is pulled into the aperture and the insert through vacuum suction, the skin is then punctured with the needles of the heat-needle insert and the heating component of the insert may also heat up the volume of tissue. As mentioned above, this may mean that the dosage of energy needed for the treatment may be less, and the lesions created in the tissue may allow for deeper and more accurate treatment as well.

In one or more embodiments, an insert containing an optical filter may be fitted into the aperture as well. In one or more embodiments, an insert containing an uncoated optic may be fitted into the aperture. Another insert may be one that has an opening on both ends or one that reduces the volume of the targeted tissue or limits the size of the area to be treated.

As mentioned above, the various modules and inserts of the device may work in combination with each other and various combinations and permutations of the above may be used by the medical personnel performing the desired treatment.

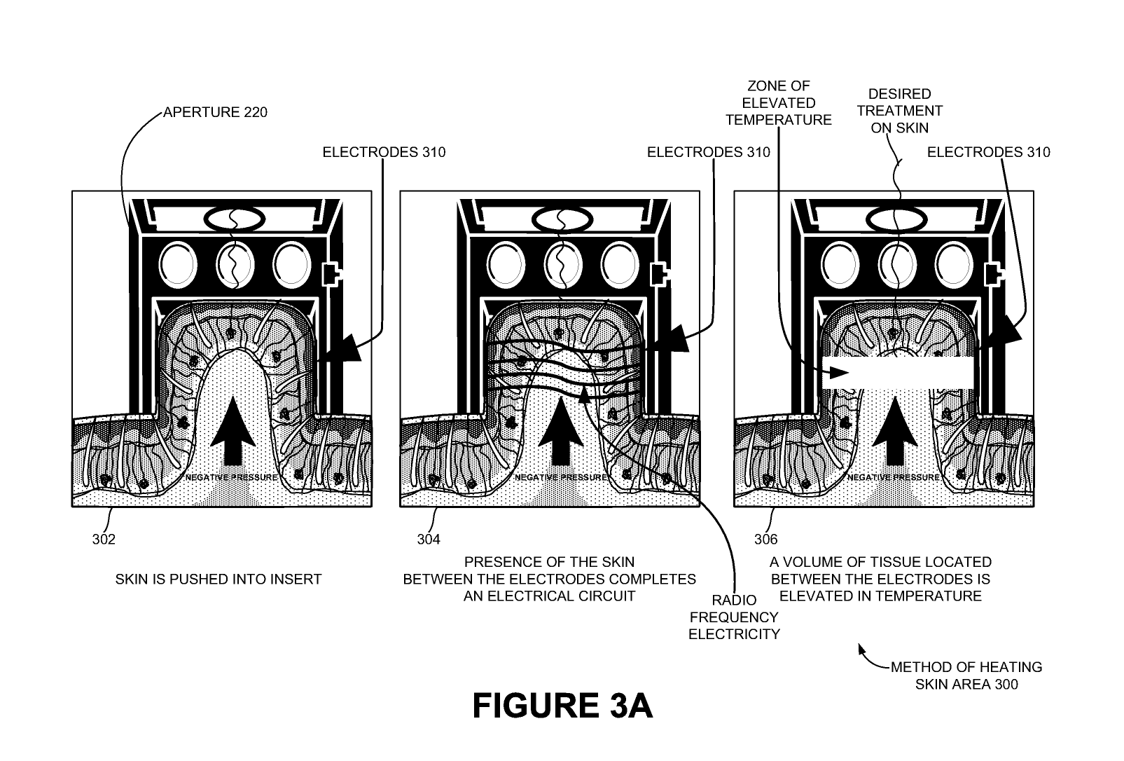

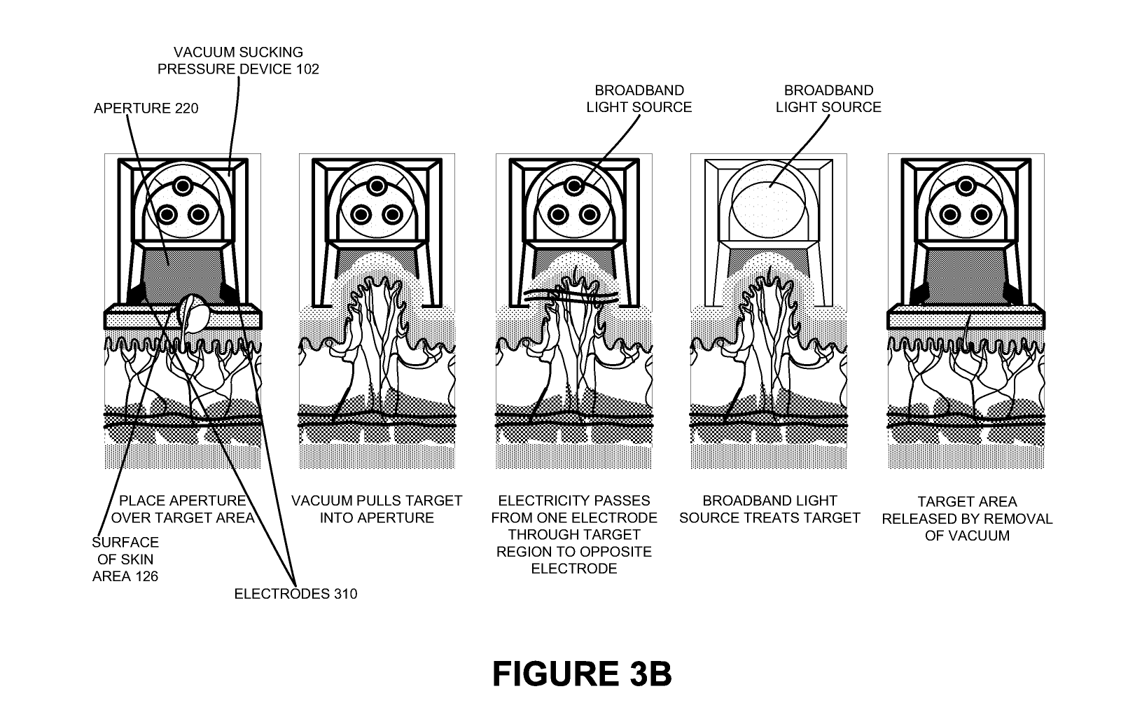

FIGS. 3A and 3B illustrate the working of the vacuum suction medical device equipped with a heat insert. FIG. 3 includes the aperture 220, the electrodes 310, the surface of the skin area, the vacuum suction pressure device 102 and the heat insert 202.

In one or more embodiments, the aperture 220 of the device 102 fitted with the heat insert 202 may be placed on the surface of the skin that is to be treated. In one or more embodiments, a vacuum suction device 102 may be applied to the surface of tissue area above a target within the tissue that is to be treated. When the vacuum is activated, the volume of tissue containing the target is pulled into the aperture where the treatment is applied. As mentioned above, the target within the tissue volume may be treated for any of the following treatments, including but not limited to acne treatment, scar removal treatment, stretch mark treatment, blemish removal treatment, cellulite reduction treatment, tattoo removal treatment, hair removal treatment and other such epidermal or dermal treatments.

In one or more embodiments, the vacuum suction module 210 of the device may be equipped with a negative pressure system and a positive pressure system that may enable the device 102 to generate a vacuum of at least 1 psi to 15 psi below atmospheric pressure within the aperture 220 of the device 102. In one or more embodiments, the device 102 may be linked to a controller, processor, circuitry and other pulse electronics. In one or more embodiments, the device 102 may further contain at least one sensor, as will be explained later. For example, the device 102 may be able to generate a vacuum pressure of a great range, and the vacuum pressure to be applied may differ based on what part of the body is being treated. In one or more embodiments, the device may be equipped with apertures of varying sizes. For example, when treating the face, an aperture of a smaller area may be used. When treating a larger area like the back, a larger aperture may be affixed to the device. In one or more embodiments, the aperture 220 may be large enough to cover at least one pilosebaceous unit of the skin

As shown in FIG. 3, the aperture of the device fitted with the heat insert may be first placed on the surface of the skin to be treated as shown in 302. The vacuum suction of the device may then be activated as shown in 304. With the activation of the vacuum, the surface of the skin area is then pulled into the insert, as shown in 304. The heat insert 202 containing the heating section may then heat up the volume of tissue pulled into the insert. In one or more embodiments, the heat may be generated by a set of electrodes 310 (or any other heating means) on the periphery of the inside section of the insert as shown in the Figure. In one or more embodiments, the heating section may raise the temperature of the tissue to a desired elevated ambient temperate. The desired elevated ambient temperature may be different for different treatments based on requirements of the desired treatment or based on the preferences of the medical personnel administering the desired treatment, in one or more embodiments. In one or more embodiments, the elevated ambient temperature may be the temperature at which the medical personnel perform the desired treatment. The normal basal body temperature for humans is 37 degree Celsius. When the tissue is pulled into the aperture of the device, the heating elements on the heat insert or the aperture may heat up the volume of tissue from the normal basal temperature of 37 C to the desired elevated ambient temperature. For example, the medical personnel may wait until the temperature of the tissue has risen to an elevated ambient temperature of 45 C. After the temperature of the tissue has risen to the elevated ambient temperature of 45 C, the medical personnel may then start the treatment (delivering the light/heat source to the skin). In one or more embodiments, the medical personnel may time the procedure such that the skin is first pulled into the aperture for a certain period of time before administering the desired treatment on the skin. For example, the medical personnel may first apply vacuum and pull up the skin into the aperture for a period of 5 seconds. After the end of the 5 seconds, the medical personnel may then start the desired treatment. At this point, the medical personnel may deliver the light or heat source to the skin for the reduced time period. For example, the tissue may be pulled into the aperture for 1 second, such that the final elevated ambient temperature of the tissue is 45 degree Celsius. After the tissue has been heated up the elevated ambient temperature, the heat generating module may then administer the heat or light to the volume of tissue. Since the ambient temperature of the skin is increased to 45 degree Celsius, the amount of heat needed to be administered to the tissue area is thereby reduced. For example, a procedure performed without the heat insert may have required an administration of broad band light to the targeted tissue area for 5 seconds. However, with the heat insert, the broad band light may only need to be administered for 2 seconds. Consequently the procedure may be more efficient and precise and may also be more cost effective. Further, the risks of damaging surrounding tissue areas is reduced because the amount of heat administered to the targeted area is also less.

After the desired amount of heat is administered to the targeted area, as shown in 304, the vacuum suction may be turned off such that the skin is released from the aperture and the insert and is restored to its normal condition, as shown in 306.

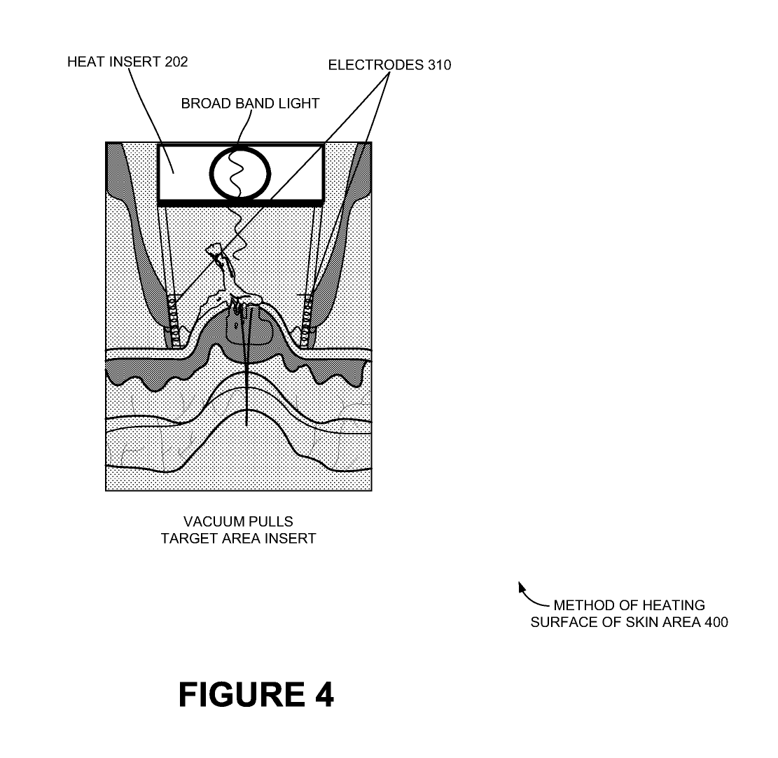

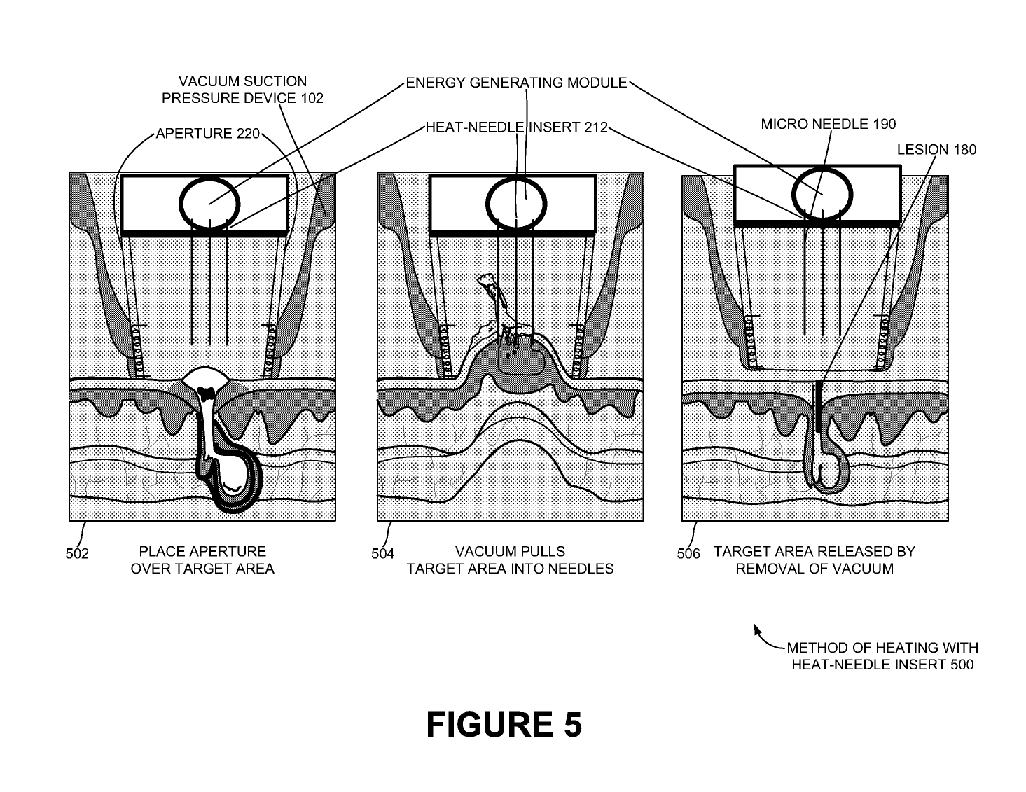

FIG. 4 is a close-up view of the insert and the aperture when the skin is pulled into the insert. FIG. 4 illustrates the heat insert 202, the electrodes 310 and a treatment using broadband light being delivered to the skin area when the vacuum suction is activated on the skin.

As mentioned above, the tissue is heated up to a higher elevated ambient temperature through the heating section of the heat insert. After the temperature of the tissue is increased to the desired elevated ambient temperature, the heating modules or the energy generating modules (broad band light module, laser module, ultrasound module etc) may then be activated to administer heat or light to the area that is pulled into the insert.

FIG. 5 is a process flow diagram of a vacuum suction pressure device equipped with a hybrid heat-needle insert. FIG. 5 shows the device 102, the heat-needle insert 214, a lesion 580 and the aperture 220.

In one or more embodiments, as mentioned above, the heat-needle insert 214 may be fitted into the aperture of the device 102. As mentioned above, the heat-needle insert 214 may have a heating component around the periphery of the inside of the insert, and may also have at least one needle recessed into the insert, as shown in 502. In such a case, the aperture fitted with the insert is first placed on the surface of the skin that is to be treated as shown in 502.

In 504, the vacuum suction is activated such a volume of tissue of the targeted area is pulled into the aperture and the insert. When the volume of tissue is pulled into the aperture 220, the needles puncture the surface of the tissue area to create lesions on the surface of the skin. Meanwhile, the heating section of the heat-needle insert increase the temperature of the tissue to the desired elevated ambient temperature. In one or more embodiments, the newly punctured and heated tissue may then be delivered light and/or heat through the heat generating modules. The presence of lesions in the skin may increase the efficiency of the treatment and the increase temperature of the tissue may reduce the amount of energy needed to perform the desired procedure. Finally, in 506, the vacuum suction may be deactivated, and the tissue is then released to its normal condition.

In one or more embodiments, the surface of the targeted tissue area may first be punctured by using a needle insert 212 over the skin first. After the skin has been treated with the needle insert 212, the needle insert 212 may be removed from the aperture 220 of the device 102, to be replaced with the heat insert 202, and the targeted tissue area may then have heat administered to the area based on the requirements of the desired procedure.

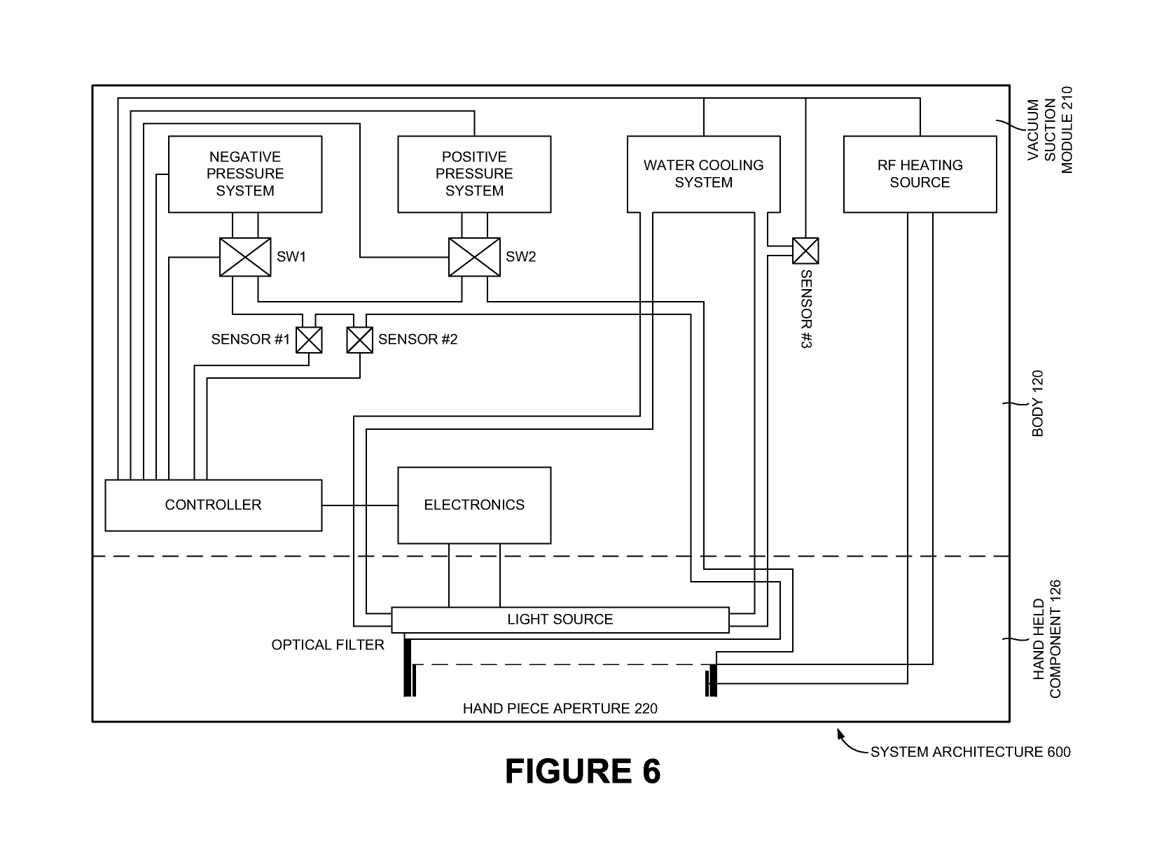

FIG. 6 illustrates the system of the device 102 further comprising a negative pressure system, a positive pressure system, a water cooling system, sensors and, a controller, a pulse electronics module. In the device 102 the treatment energy is provided by light filtered by optical filter and the heating elements use radio frequency energy. Treatment is provided using hand piece aperture 220. Part of the modules are in the body and the light source and the aperture are located in the hand-held component 126.

In one or more embodiments, the medical device, in combination with various modules and inserts, as described above, may be used for a variety of treatment procedures. Some of the treatments are described below.

Laser or Broadband Light Hair Removal

Medical treatments performed by supplying energy through the epidermis may heat the target to a temperature sufficient to destroy the target without destroying or damaging the surrounding tissue or surface of the skin. For example, a patient may seek treatment for the removal of unwanted hair. In one or more embodiments, medical personnel may apply a light based device over the area to be treated and, using the light, heat a targeted hair follicle to a temperature sufficient to destroy it. This temperature is often in excess of 60 C. The targeted hair follicle is thus heated from its basal temperature of approximately 37 C to a temperature in excess of 60 C. Accomplishing this increase in temperature requires a certain amount of light energy. A portion of the applied energy is absorbed in parts of the tissue other than the target and may heat those parts to a destructive temperature. Often those parts of tissue heated to a destructive temperature are in the epidermis where melanin is a strong absorber of light.

In one or more embodiments, the heat insert shown (202) in FIG. 3 can be used to preheat the volume of tissue containing the target to a temperature above the basal temperature. In one or more embodiment, the heating elements may heat the volume of tissue containing the target to an elevated ambient temperature of 45 C. After the volume of tissue has been heated, the desired type of light(broad band or laser) may then be delivered to the skin through the aperture such that final temperature of the skin then rises from 45 C to over 60 C rather than from the basal body temperature of 37 C. Less light energy is required to heat the target from a starting temperature of 45 C compared to a starting temperature of 37 C. If the same procedure was performed without the heat insert, more light and heat may need to be delivered to the tissue to raise the temperature from 37 C to 60 C. Since high intensity light could potentially be harmful to the epidermis and dermis. The use of the heat insert significantly helps reduce excessive exposure to light, that in turn, may help prevent discoloration of the skin such as hypopigmentation or hyperpigmentation or the burning of the epidermis. Since the objective of the light is mainly to increase the temperature of the target, the heat insert provides an effective way to reach part way to the desired temperature, and also conserves light and heat energy at the same time.

Collagen Treatment

Medical treatments performed by supplying energy through the epidermis may heat the target to a temperature sufficient to destroy the target without destroying or damaging the surrounding tissue or surface of the skin. For example, a patient may seek treatment for skin resurfacing. Medical personnel may apply a light based device over the area to be treated and, using the light, heat targeted collagen to a temperature sufficient to denature it. This temperature is often in excess of 60 C. The targeted collagen is thus heated from its basal temperature of approximately 37 C to a temperature in excess of 60 C. Accomplishing this increase in temperature requires a certain amount of light energy. A portion of the applied energy is absorbed in parts of the tissue other than the target and may heat those parts to a destructive temperature. Often those parts of tissue heated to a destructive temperature are in the epidermis where melanin is a strong absorber of light. Accomplishing this increase in temperature requires a certain amount of radio frequency energy.

Using the heat insert, as was the case with laser hair removal, less light may be applied to the tissue in order to raise the target's temperature to the desired temperature because the tissue may have already reached half the target temperature through the heat insert. Therefore, the skin's exposure to the light is reduced, decreasing the chance of hyperpigmentation or hypopigmentation or the burning of the epidermis. Since the objective of the light is mainly to increase the temperature of the target, the heating element provides an effective way to reach part way to the desired temperature, and conserving light and energy at the same time.

Acne Treatment

Another common procedure frequently administered through the skin is treatment for acne conditions. For example, a patient may seek treatment for removal of acne lesions. Medical personnel may apply a light based device over the area to be treated and, using the light, heat the targeted pilosebaceous unit to a temperature sufficient to clear the acne lesion. This temperature is often in excess of 60 C. The targeted pilosebaceous unit is thus heated from its basal temperature of approximately 37 C to a temperature in excess of 60 C. Accomplishing this increase in temperature requires a certain amount of light energy. A portion of the applied energy is absorbed in parts of the tissue other than the target and may heat those parts to a destructive temperature. Often those parts of tissue heated to a destructive temperature are in the epidermis where melanin is a strong absorber of light.

Using the heat insert, as was the case with laser hair removal, less light may be applied to the tissue in order raise the target's temperature to the desired temperature because the tissue may have already reached half the target temperature through the heat insert Therefore, the skin's exposure to the light is therefore reduced, decreasing the chance of hyperpigmentation or hypopigmentation or the burning of the epidermis. Since the objective of the light is mainly to increase the temperature of the target, the heating element provides an effective way to reach part way to the desired temperature, and conserving light and energy at the same time.

In one or more embodiments, the medical personnel may use the needle insert to create lesions on the surface of the skin area as described above and to penetrate the pilosebaceous unit and draw the sebum out using either vacuum or other form of pressure. In one or more embodiments, the heating elements shown (202) in FIG. 3 can be used to preheat the volume of tissue containing the pilosebaceous unit to a temperature above the basal temperature while the needle is inserted. In one or more embodiment, the heating elements may heat the volume of tissue containing the target to an elevated ambient temperature of 45 C.

Fat Destruction

Medical treatments performed by supplying energy through the epidermis may heat the target to a temperature sufficient to destroy the target without destroying or damaging the surrounding tissue or surface of the skin. For example, a patient may seek treatment for fat reduction. Medical personnel will apply a ultrasonic device into the area to be treated and, using ultrasonic energy, heat targeted fat cells to a temperature sufficient to destroy them. This temperature is often in excess of 60 C. The targeted fat cells is thus heated from its basal temperature of approximately 37 C to a temperature in excess of 60 C. Accomplishing this increase in temperature requires a certain amount of ultrasound energy. A portion of the applied energy is absorbed in parts of the tissue other than the target and may heat those parts to a destructive temperature.

In one or more embodiment, the heating elements shown in FIG. 2 can be used to preheat the volume of tissue containing the target to a temperature above the basal temperature. In one or more embodiment, the heating elements may heat the volume of tissue containing the target to an elevated ambient temperature of 45 C. When the ultrasound energy is applied to the tissue, the target temperature must rise from 45 C to over 60 C rather than from the basal body temperature of 37 C. Less ultrasound energy is required to heat the target from a starting temperature of 45 C compared to a starting temperature of 37 C. Since high intensity ultrasound could potentially be harmful to the epidermis and dermis, this device significantly helps reduce excessive exposure to ultrasound, that in turn, may help prevent the burning of the epidermis and dermis.

Additional Uses

In one or more embodiments, the medical personnel may gently place the aperture 220 containing the heat insert over the target to be treated. In one or more embodiments, the medical personnel may turn the device 102 on, such that a vacuum pressure is applied through the aperture 220 of the device 102. In one or more embodiments, when the vacuum pressure is applied, the underlying skin may be pulled into the aperture 220 of the device 102 as shown in FIG. 1. In one or more embodiments, when the skin is pulled into the aperture of the device 102, the heating elements may heat up all or a portion of the volume of tissue with the heating elements. The tissue volume pulled into the aperture may thus be heated up from the basal body temperature of 37 C to an elevated ambient temperature of 45 s Celsius, in one or more embodiments. In one or more embodiments, the vacuum pressure may still be intact, and the surface of the skin area may then be exposed to broad band or other light therapy that may be directly shone onto the middle part of the skin that is pulled up due to the vacuum. In one or more embodiments, the vacuum pressure may still be intact, and the surface of the skin area may then be exposed to ultrasound therapy that may be directly applied onto the surface of the skin that is pushed into the aperture due to the vacuum. In one or more embodiments, the vacuum pressure may still be intact, and the surface of the skin area may then be penetrated by a needle or an array of needles and radio frequency energy may or may not be applied through the needles. Since the volume of tissue inside the aperture area is already heated up to 45 C or higher, the amount of energy needed to heat up the target may be less than what would have been required if the tissue had not already been heated up.

Other Ways to Use the Device

In one or more embodiments, the medical personnel the medical personnel may gently place the aperture 220 of the device 102 over the target to be heated. Inside aperture 220 is a heating element. In one or more embodiments, the medical personnel may turn the device 102 on, such that a vacuum pressure is applied through the aperture 220 of the device 102. In one or more embodiments, when the vacuum pressure is applied, the underlying skin may be pulled into the aperture 220 of the device 102 as shown in FIG. 3. In one or more embodiments, when the skin is pulled into the aperture of the device 102, the heating elements may heat up all or a portion of the volume of tissue that is in direct contact with the heating elements. The tissue volume pulled into the aperture may thus be heated up to the ambient temperature of 45 degree Celsius, in one or more embodiments. In one or more embodiments, the vacuum pressure may not be still intact and the volume of tissue has been released from the aperture, and the surface of the skin area may then be exposed to broad band or other light therapy that may be directly shone onto the middle part of the skin that is pulled up due to the vacuum. In one or more embodiments, the vacuum pressure may not be still intact and the volume of tissue has been released from the aperture, and the surface of the skin area may then be exposed to ultrasound therapy that may be directly applied onto the surface of the skin that is pushed into the aperture due to the vacuum. In one or more embodiments, the vacuum pressure may not be still intact and the volume of tissue has been released from the aperture, and the surface of the skin area may then be penetrated by a needle or an array of needles and radio frequency energy may be applied through the needles. Since the volume of tissue that was inside the aperture area is already heated up to 45 C or higher, the amount of energy needed to heat up the target may be less than what would have been required if the tissue had not already been heated up.

In one or more embodiments, the medical personnel may go through an entire light treatment on the area to be treated. In one or more embodiments, the medical personnel may remove the insert and simply use light therapy on the recently heated and punctured tissue area. In another embodiments, the skin may be punctured with a needle insert first before the treatment begins, and the medical personnel may then remove the needle insert and use the heat insert that may then heat up the surface of the skin area, and may subsequently apply the light therapy on the targeted area. In one or more embodiments, when the needle insert is used first, the medical personnel may go through the entire targeted area with the needle insert and may apply pressure uniformly throughout the treatment area through the needle insert 103 such that lesions are uniformly created on the surface of the skin.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware, firmware and software (e.g., embodied in a machine readable medium). For example, the various electrical structure and methods may be embodied using transistors, logic gates, and electrical circuits (e.g., application specific integrated (ASIC) circuitry and/or in digital signal processor (DSP) circuitry).

In addition, it will be appreciated that the various operations, processes, and methods disclosed herein may be embodied in a machine-readable medium and/or a machine accessible medium compatible with a data processing system (e.g., a computer devices), may be performed in any order (e.g., including using means for achieving the various operations). Accordingly, the specification and drawings are to be regarded in an illustrative rather than a restrictive sense.

What is claimed is:

1. A method comprising:
placing a device enabled with vacuum suction pressure on a surface of a tissue area to be treated;
applying a vacuum suction pressure on the surface of the skin area to pull up the skin area and an underlying tissue into an aperture opening of the device;
simultaneously, while retaining vacuum suction, heating up a volume of tissue that is pulled up inside the aperture opening of the device such that the temperature of the tissue area rises to an elevated ambient temperature; and
performing a desired treatment, through an energy-generating module, on the tissue volume after the tissue area has been heated up.

2. The method of claim 1 further comprising:
inserting a heat insert into the aperture of the device,
wherein the heat insert contains a set of heating elements on the periphery of the inside section of the heat insert to heat up the volume of tissue that is pulled up inside the aperture of the device.

3. The method of claim 2 wherein the heating elements is at least one of a set of electrodes connected to a radio frequency energy source

4. The method of claim 1 wherein the aperture of the device contains a set of heating elements on the periphery of the inside section of the aperture to heat up the volume of tissue that is pulled up inside the aperture of the device.

5. The method of claim 4 wherein the heating elements is at least one of a set of electrodes, is connected to a radio frequency source.

6. The method of claim 1 further comprising:
releasing the application of the vacuum suction pressure on the surface of the tissue area at a time that marks at least one of before a start of the desired treatment and a completion of the desired treatment.

7. The method of claim 1 further comprising:
delivering at least one of a light and a source of heat energy, through the energy-generating module, to the tissue area to after the volume of tissue after the temperature of the tissue has risen to the elevated ambient temperature from the normal body basal temperature.

8. The method of claim 7 wherein the at least one of a light and a source of heat energy is at least one of a broad band light, a laser light, a radiofrequency energy and an ultrasound light.

9. The method of claim 1 wherein the desired treatment is for at least one of an acne treatment, an acne scar treatment, a cellulite treatment, a stretch mark treatment, a fat removal treatment, a vascular lesion removal treatment, a hair removal treatment and a tattoo removal treatment.

10. The method of claim 1 further comprising:
inserting a heat-needle insert into the aperture of the device, wherein the heat needle insert contains at least one needle recessed into the insert and a set of heating elements on the periphery of the inside section of the insert;
when the vacuum suction pressure is applied to the surface of the skin, puncturing the surface of the tissue area with the at least one needle recessed into the heat-needle insert to create at least one lesion in the tissue to be treated;
when the vacuum suction pressure is applied to the surface of the skin, heating up the volume of the tissue that is pulled up into the aperture of the device through the heating elements of the heat-needle insert; and
applying the desired treatment on the tissue containing the lesion after the temperature of the tissue volume has risen to the elevated ambient temperature.

11. The method of claim 1 wherein the aperture of the device contains at least one needle recessed into the aperture to puncture the surface of the tissue area.

12. The method of claim 1 wherein an energy required for effective clinical outcome from the desired treatment is reduced because the tissue volume is already heated to the elevated ambient temperature that is higher than the resting basal temperature of the tissue volume.

13. An apparatus comprising:
a device enabled with vacuum suction pressure:
to enclose a volume of tissue area that is to undergo a desired treatment,
to pull in the volume of the tissue area into an aperture of the device when vacuum suction pressure is applied,
to release an application of the vacuum suction pressure at a time that marks at least one of before a start of the desired treatment and a completion of the desired treatment, and
a set of heating elements:
to simultaneously heat up the volume of tissue that is pulled inside the aperture such that the temperature of the tissue area rises to an elevated ambient temperature, and
an energy generating module to:
deliver at least one of a light and a source of heat energy to the tissue area to after the volume of tissue after the temperature of the tissue has risen to the elevated ambient temperature from a normal body basal temperature.

14. The apparatus of claim 13 wherein the set of heating elements is located on the periphery of at least one of an inside section of the aperture of the device and an inside section of a heat insert that is inserted into the aperture of the device.

15. The apparatus of claim 13 wherein the heating elements is at least one of a set of electrodes connected to a radio frequency emitter.

16. The apparatus of claim 13 wherein the at least one of a light and a source of heat energy is at least one of a broad band light, a laser light, a radiofrequency energy and an ultrasound light.

17. The apparatus of claim 13 wherein the desired treatment is for at least one of an acne treatment, an acne scar treatment, a cellulite treatment, a stretch mark treatment, a fat removal treatment, a vascular lesion removal treatment, a hair removal treatment and a tattoo removal treatment.

18. The apparatus of claim 13 further comprising:
a heat-needle insert, wherein the heat needle insert contains at least one needle recessed into the insert and a set of heating elements on the periphery of the inside section of the insert:
to puncture the tissue area pulled into the aperture and to create at least one lesion on the surface of the tissue.

19. The apparatus of claim 13 wherein an energy required for effective clinical outcome from the desired treatment is reduced because the tissue volume is already heated to the elevated ambient temperature that is higher than the resting basal temperature of the tissue volume.

20. A method comprising:
applying a vacuum suction pressure on the surface of the skin area to pull up the skin area and an underlying tissue into an aperture opening of the device;
simultaneously, while retaining vacuum suction, heating up a volume of tissue that is pulled up inside the aperture opening of the device such that the temperature of the tissue area rises to an elevated ambient temperature;
performing a desired treatment, through an energy-generating module, on the tissue volume after the tissue area has been heated up; and
releasing the application of the vacuum suction pressure on the surface of the tissue area at a time that marks at least one of before a start of the desired treatment and a completion of the desired treatment.

21. The method of claim 20 further comprising:
inserting a heat-needle insert into the aperture of the device, wherein the heat needle insert contains at least one needle recessed into the insert and a set of heating elements on the periphery of the inside section of the insert;
when the vacuum suction pressure is applied to the surface of the skin, puncturing the surface of the tissue area with the at least one needle recessed into the heat-needle insert to create at least one lesion in the tissue to be treated;
when the vacuum suction pressure is applied to the surface of the skin, heating up the volume of the tissue that is pulled up into the aperture of the device through the heating elements of the heat-needle insert; and
applying the desired treatment on the tissue containing the lesion after the temperature of the tissue volume has risen to the elevated ambient temperature.

22. The method of claim 20 further comprising:
delivering at least one of a light and a source of heat energy, through the energy-generating module, to the tissue area to after the volume of tissue after the temperature of the tissue has risen to the elevated ambient temperature from the normal body basal temperature,
wherein the at least one of a light and a source of heat energy is at least one of a broad band light, a laser light, a radiofrequency energy and an ultrasound light.

23. The method of claim 20 wherein the desired treatment is for at least one of an acne treatment, an acne scar treatment, a cellulite treatment, a stretch mark treatment, a fat removal treatment, a vascular lesion removal treatment, a hair removal treatment and a tattoo removal treatment.
